(19) United States
(12) Patent Application Publication
(10) Pub. No.: US 2015/0059019 A1
(43) Pub. Date: Feb. 26, 2015

(54) AGRONOMIC CHARACTERISTICS OF PLANTS THROUGH ABPH2

(71) Applicants: E I DU PONT DE NEMOURS AND COMPANY, Wilmington, DE (US); COLD SPRING HARBOR LABORATORY, COLD SPRING HARBOR, NY (US)

(72) Inventors: David Peter Jackson, New York, NY (US); Stephen M. Allen, Wilmington, DE (US); Robyn Johnston, Cold Spring Harbor, NY (US); Victor Llaca, Newark, DE (US); Fang Yang, Cold Spring Harbor, NY (US)

(21) Appl. No.: 14/384,698

(22) PCT Filed: Mar. 13, 2013
(86) PCT No.: PCT/US2013/030635

§ 371 Date: Sep. 12, 2014

Related U.S. Application Data
(60) Provisional application No. 61/610,690, filed on Mar. 14, 2012.

(51) Int. Cl. C12N 15/82; C07K 14/415; C12Q 1/68

(52) U.S. Cl. CPC C12N 15/8261; C12Q 1/6895; C07K 14/415; C12N 15/8213; C12Q 2600/156; C12Q 2600/13
USPC 800/278; 435/6.11; 800/298; 800/317.4; 800/320.1; 800/312; 800/322; 800/320; 800/306; 800/320.3; 800/314; 800/320.2; 536/23.6

(57) ABSTRACT

Methods and compositions for modulating an agronomic characteristic of a plant are provided. Methods are provided for modulating the expression of Abph2 sequence in a host plant or plant cell to modulate agronomic characteristics such as altered ear number and increased yield.

### CROSS REFERENCE TO RELATED APPLICATIONS

This application claims the benefit of U.S. Provisional Application No. 61/610,690, filed Mar. 14, 2012, the entire content of which is herein incorporated by reference.

### GOVERNMENT SUPPORT

The invention described herein was made in whole or in part with government support under United States Department of Agriculture Grant No. 2011-67013-30031 awarded by the National Institute of Food and Agriculture under the Agriculture and Food Research Initiative Competitive Grants Program. The United States Government has certain rights in the invention.

### FIELD

The disclosure relates to the field of the improving crop performance,

### BACKGROUND

Plant morphology and diversity are largely dependent on the establishment of phyllotaxy, which is initiated from a group of stem cells in the shoot apical meristem (SAM). Leaves and the axillary meristems that generate branches and flowers are initiated in regular patterns from the shoot apical meristem (SAM). The cells of the shoot apical meristem summit serve as stem cells that divide to continuously displace daughter cells to the surrounding regions, where they are incorporated into differentiated leaf or flower primordia. The meristems are thus capable of regulating their size during development by balancing cell proliferation with the incorporation of cells into new primordia. The SAM provides all aerial parts of plant body.

In a decussate (opposite) pattern, leaves are arranged along the stem in opposite pairs, with each successive pair oriented at 90 degrees. For example, Example, Cyprus has decussate pattern. In a distichous (alternate) pattern, single leaves alternate on either side of the stem. For example, maize has alternate phyllotaxy. In spiral phyllotaxy, single leaves are offset by an angle of about 137.5 degrees. Example includes Arabidopsis and other plants. In plants phyllotaxy can change during development. In maize, the main leaves on the stem are arranged in alternate phyllotaxy as mentioned above, whereas, the husks on the ear are arranged in a spiral phyllotaxy.

Auxin is an important factor controlling phyllotactic patterns. Studies on a phyllotaxy mutant in maize have shown that cytokinin, as well as its crosstalk with auxin, play an important role in this process.

The central concept of stem cells regulation is known by the signal pathway of CLAVATA/WUSCHEL (CLV/WUS) genes. Loss of CLV1, CLV2, or CLV3 activity in Arabidopsis causes accumulation of undifferentiated cells in the shoot apex, indicating that CLV genes together promote the timely transition of stem cells into differentiation pathways, or repress stem cell division, or both (Fletcher et al. (1999) Science 283:1911-1914; Taguchi-Shiobare et al. (2001) Genes and Development 15:2755-5766; and, Trotochaud et al. (1999) Plant Cell 11:393-405; Merton et al. (1954) Am. J. Bot. 41:726-32 and Szymkowiak et al. (1992) Plant Cell 4:1089-100; Yamamoto et al. (2000) Biochim. Biophys. Acta. 1491:333-40). The maize orthologues of CLV1/2 are TD1 and FEA2, that have been reported (Taguchi-Shiobara et al. (2001) Genes Dev. 65 15:2755-66). It is desirable to be able to control the size and appearance of shoot and floral meristems, to give increased yields of leaves, flowers, and fruit. Accordingly, it is an object of the invention to provide novel methods and compositions for the modulation of meristem development.

Modulating phyllotaxy and the inflorescence development play important roles in improving agronomic performance of crop plants.

### SUMMARY

In an embodiment, the disclosure provides a method of producing a transgenic plant with modulated expression of Abph2, the method comprising the steps of (a) introducing into a regenerable plant cell a recombinant construct comprising a polynucleotide sequence operably linked to a promoter, wherein the expression of the polynucleotide sequence modulates Abph2 expression; (b) regenerating a transgenic plant from the regenerable plant cell after step (a), wherein the transgenic plant comprises in its genome the recombinant DNA construct; and (c) selecting a transgenic plant of (b), wherein the transgenic plant comprises the recombinant DNA construct and exhibits modulated expression of Abph2, when compared to a control plant not comprising the recombinant DNA construct.

A method of producing a transgenic plant with modulated expression of Abph2, the method includes (a) introducing into a regenerable plant cell a recombinant construct comprising a polynucleotide operably linked to a promoter, wherein the expression of the polynucleotide sequence modulates Abph2 expression or activity; (b) regenerating a transgenic plant from the regenerable plant cell after step (a), wherein the transgenic plant comprises in its genome the recombinant DNA construct; and (c) selecting the transgenic plant of (b), wherein the transgenic plant comprises the recombinant construct and exhibits an alteration in the expression of Abph2, when compared to a control plant not comprising the recombinant DNA construct.

A method of producing a transgenic plant with modulated expression of Abph2, the method includes modulating the expression of polynucleotide encoding the amino acid sequence of SEQ ID NO; 1 or a sequence that is at least 70% identical to SEQ ID NO: 1.

A method of producing a transgenic plant with modulated expression of Abph2, the method includes modulating the expression of a polynucleotide encoding the amino acid sequence selected from the group consisting of SEQ ID NOS: 1 and 6-31, a functional domain thereof, and a sequence that is at least 70% identical to SEQ ID NOS: 1 and 6-31.

A method of increasing yield of a maize plant, the method includes transgenically altering the expression of Abph2 gene such that the number of ears harvested per maize plant is increased relative to a maize plant that is not transgenically altered as such.

### BRIEF DESCRIPTION OF THE DRAWINGS AND SEQUENCE LISTINGS

The disclosure can be more fully understood from the following detailed description and the accompanying drawings and Sequence Listing which form a part of this application. The Sequence Listing contains the one letter code for nucleotide sequence characters and the three letter codes for amino acids as defined in conformity with the IUPAC-IUBMB standards described in Nucleic Acids Research 13:3021-3030 (1985) and in the Biochemical Journal 219 (No. 2); 345-373 (1984), which are herein incorporated by reference in their entirety. The symbols and format used for nucleotide and amino acid sequence data comply with the rules set forth in 37 C.F.R. §1.822.

FIG. 1A shows that Abph2 mutant plants have opposite and decussate leaves after about 5th leaf and that shoot meristem is wider than the wild-type plants. In some genetic backgrounds, Abph2 plants develop multiple shoots. FIGS. 1B and 1C show that shoot meristem is wider in ABPH2 plants.

FIG. 2 shows the insertion region of Abph2 and the map-based cloning approach to isolate the Abph2-allele.

FIG. 3 shows possible translocation of the Abph2 locus to a new chromosomal location from its original location on chromosome 7. The approximate chromosomal distance between the original and the translocated position is about 800 kb. “GRX” denotes the glutaredoxin gene. “BRF” denotes the Branch super family gene.

FIG. 4A shows that a targeted EMS knockout screen was used to develop two independent Abph2 phenotypic revertants that have mutations in the glutaredoxin (GRX) gene. FIG. 4B and FIG. 40 show the two independent mutations V65M and C75T, respectively.

FIG. 5A shows that pAbph2::ABPH2-YFP transgenics phenocopy Abph2A and confirmed by the fluorescence imaging (FIG. 5B).

FIG. 6A shows the expression pattern of Abph2 in leaf primordial compared to the wild-type plants. Abph2 expression pattern in anthers is shown in FIG. 6B.

FIG. 7A shows a model of how ABPH2 (GRX) and FEA4 (bZIP) interact in the nucleus. FIG. 7B shows the Interaction of FEA4 and ABPH2 by bimolecular fluorescence complementation (nYFP-ABPH2+cYFP-FEA4). FIG. 7C is a negative control (nYFP-AS1+cYFP-FEA4).

FIG. 8 shows that ABPH2 (GRX) and FEA4 (bZIP interaction factor) interact via yeast 2 hybrid interaction. SD/−LW synthetic dropout media minus Leucine and tryptophan; SD/−AHLW synthetic dropout media minus adenine histidine Leucine Tryptophan.

FIG. 9 shows a schematic illustration of a pathway regulating meristem size and shows the functional interaction of ABPH2 and FEA4.

The sequence descriptions (Table 1) and Sequence Listing attached hereto comply with the rules governing nucleotide and/or amino add sequence disclosures in patent applications as set forth in 37 C.F.R. §1.821-1.825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (2):345-373 (1984) which are herein incorporated by reference. The symbols and format used for nucleotide and amino acid sequence data comply with the rules set forth in 37 C.F.R. §1.822.

TABLE 1

| | | SEQ |
|---|---|---|
| Description | Type* | ID NO: |
| ABPH2 protein | PP | 1 |
| Abph2 coding region | PN | 2 |
| Abphyl2-EMS knockout allele 1 (V65M) | PN | 3 |
| Abphyl2-EMS knockout allele 2 (C75T) | PN | 4 |
| 4.5 kb insertion in Abph2-0 dominant mutant | PN | 5 |
| Oryza sativa | PP | 6 |
| Sorghum bicolor | PP | 7 |
| Arabidopsis At5g14070.1 | PP | 8 |
| Arabidopsis At3g02000.1 | PP | 9 |
| Arabidopsis At4g15700.1 | PP | 10 |
| Arabidopsis At4g15690.1 | PP | 11 |
| Soybean 16g05730.1 | PP | 12 |
| Soybean 19g26770.1 | PP | 13 |
| Sorghum bicolor 04g020790.1 | PP | 14 |
| Osmunda cinnamomea (Cinnamon fern) Locus_13954 | PP | 15 |
| Maize predicted | PP | 16 |
| Clone fds1n.pk018.j5; Momordica charantia | PP | 17 |
| Clone evl2c.pk006.b5; Viola soraria | PP | 18 |
| Clone ecl1c.pk008.m12; Nepeta racemosa | PP | 19 |
| Clone hengr1n.pk013.a4_1; Lamium amplexicaule | PP | 20 |
| Clone hengr1n.pk069.m2_1; Lamium amplexicaule | PP | 21 |
| Clone hengr1n.pk085.g8_1; Lamium amplexicaule | PP | 22 |
| Clone arttr1n.pk067.c3_1; Artemisia tridentate | PP | 23 |
| Clone arttr1n.pk049.m7_1; Artemisia tridentate | PP | 24 |
| Clone ahgr1c.pk230.c22_1; Amaranthus | PP | 25 |
| hypochondriacus | | |
| Clone ahgr1c.pk069.o1_1; Amaranthus | PP | 26 |
| hypochondriacus | | |
| Clone ahgr1c.pk213.b1_1; Amaranthus | PP | 27 |
| hypochondriacus | | |
| Clone sesgr1n.pk102.h21_1; Sesbania bispinosa | PP | 28 |
| Clone ehsf2n.pk162.c19_1; Dennstaedtia | PP | 29 |
| punctilobula | | |
| Clone ehsf2n.pk037.b19_1; Dennstaedtia | PP | 30 |
| punctilobula | | |
| Clone epn2n.pk040.c2_1; Paspalum notatum | PP | 31 |

*Polynucleotide (PN); Polypeptide (PP)

The sequence descriptions and Sequence Listing attached hereto comply with the rules governing nucleotide and/or amino acid sequence disclosures in patent applications as set forth in 37 C.F.R. §1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 4:345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

The terms “monocot” and “monocotyledonous plant” are used interchangeably herein. A monocot of the disclosure includes the Gramineae.

The terms “dicot” and “dicotyledonous plant” are used interchangeably herein. A dicot of the disclosure includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

The terms “Abph2” and “Abhyl2” are used interchangeably herein.

“Modulated expression of Abph2” or “modulating the expression of Abph2” or “altered/altering the expression of Abph2” generally refers to a change in one or more of the expression parameters such as strength (magnitude), specificity (e.g., tissue specificity), and temporal (timing—i.e., during embryogenesis). In addition, such modulation or alteration can also be made by a change in the amino acid sequence of Abph2 such that its activity is affected. In addition, by affecting regulatory elements of endogenous Abph2 gene, one can modulate the expression and/or activity of Abph2.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, ear meristem size, tassel size, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from an mRNA template using the enzyme reverse transcriptase. The cDNA can be single-stranded or converted into the double-stranded form using the Klenow fragment of DNA polymerase I.

“Coding region” refers to a polynucleotide sequence that when transcribed, processed, and/or translated results in the production of a polypeptide sequence.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

‘Isolated’ refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

“Regulatory sequences” or “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Overexpression” refers to the production of a gene product in transgenic organisms that exceeds levels of production in a null segregating (or non-transgenic) organism from the same experiment.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

A “favorable allele” is the allele at a particular locus that confers, or contributes to, a desirable phenotype, e.g., increased cell wall digestibility, or alternatively, is an allele that allows the identification of plants with decreased cell wall digestibility that can be removed from a breeding program or planting (“counterselection”). A favorable allele of a marker is a marker allele that segregates with the favorable phenotype, or alternatively, segregates with the unfavorable plant phenotype, therefore providing the benefit of identifying plants.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes. Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

The term “locus” generally refers to a genetically defined region of a chromosome carrying a gene or, possibly, two or more genes so closely linked that genetically they behave as a single locus responsible for a phenotype. When used herein with respect to Abph2, the “Abph2 locus” shall refer to the defined region of the chromosome carrying the Abph2 gene including its associated regulatory sequences, plus the region surrounding the Abph2 gene that is non colinear with B73, or any smaller portion thereof that retains the Abph2 gene and associated regulatory sequences.

A “gene” shall refer to a specific genetic coding region within a locus, including its associated regulatory sequences. One of ordinary skill in the art would understand that the associated regulatory sequences will be within a distance of about 4 kb from the Abph2 coding sequence, with the promoter located upstream.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells, that can be cultured into a whole plant.

After alignment of the sequences, using the Clustal W program, it is possible to obtain “percent identity” and “divergence” values by viewing the “sequence distances” table on the same program; unless stated otherwise, percent identities and divergences provided and claimed herein were calculated in this manner.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

It is well understood by those skilled in the art that different promoters may direct the expression of a gene in different tissues or cell types, or at different stages of development, or in response to different environmental conditions.

Promoters that can be used for this disclosure include, but are not limited to, shoot apical meristem specific promoters. Maize knotted 1 promoter, and promoters from genes that are known to be expressed in maize SAM can be used for expressing the polynucleotides disclosed in the disclosure. Examples of such genes include, but are not limited to Zm phahulosa, terminal ear1, rough sheath2, rolled leaf1, zyb14, narrow sheath (Ohtsu, K. at al (2007) Plant Journal 52, 391-404). Promoters from orthologs of these genes from other species can be also be used for the disclosure.

Examples of Arabidopsis promoters from genes with SAM-preferred expression include, but are not limited to, clv3, aintegumenta-like (ail5, ail6, and ail7) and terminal ear like1, clavata1, wus, shootmeristemless, terminal flower1 (Yadav et al (2009) Proc Natl Acad Sci USA. March 24).

PCT Publication Nos. WO 2004/071467 and U.S. Pat. No. 7,129,089 describe the synthesis of multiple promoter/gene/terminator cassette combinations by ligating individual promoters, genes, and transcription terminators together in unique combinations. Generally, a NotI site flanked by the suitable promoter is used to clone the desired gene. NotI sites can be added to a gene of interest using FOR amplification with oligonucleotides designed to introduce NotI sites at the 5′ and 3′ ends of the gene. The resulting FOR product is then digested with NotI and cloned into a suitable promoter/NotI/terminator cassette. Although gene cloning into expression cassettes is often done using the NotI restriction enzyme, one skilled in the art can appreciate that a number of restriction enzymes can be utilized to achieve the desired cassette. Further, one skilled in the art will appreciate that other cloning techniques including, but not limited to, PCR-based or recombination-based techniques can be used to generate suitable expression cassettes.

In addition, WO 2004/071467 and U.S. Pat. No. 7,129,089 describe the further linking together of individual promoter/gene/transcription terminator cassettes in unique combinations and orientations, along with suitable selectable marker cassettes, in order to obtain the desired phenotypic expression. Although this is done mainly using different restriction enzymes sites, one skilled in the art can appreciate that a number of techniques can be utilized to achieve the desired promoter/gene/transcription terminator combination or orientations. In so doing, any combination and orientation of shoot apical meristem-specific promoter/gene/transcription terminator cassettes can be achieved. One skilled in the art can also appreciate that these cassettes can be located on individual DNA fragments or on multiple fragments where co-expression of genes is the outcome of co-transformation of multiple DNA fragments.

Plants with Abph2 mutations, wherein the mutation results in a gain of Abph2 function or modulation of Abph2 expression are also called “Abph2 plants” or “Abph2 null plants”.

Plants with weak Abph2 mutations, wherein the mutation results in varying degree of Abph2 function or modulation of Abph2 expression are also called “Abph2 plants with weak Abph2 phenotype”. “Weak Abph2 alleles” as referred to herein are Abph2 variants or variants of SEQ ID NOS: 1 or 6-31, which confer weak Abph2 phenotype on the plant.

The term “dominant negative mutation” as used herein refers to a mutation that has an altered gene product that acts antagonistically to the wild-type allele. These mutations usually result in an altered molecular function (often inactive) and are characterized by a “dominant negative” phenotype. A gene variant, a mutated gene or an allele that confers “dominant negative phenotype” would confer a “null” or a “mutated” phenotype on the host cell even in the presence of a wild-type allele. As used herein, a polypeptide (or polynucleotide) with “Abph2 activity” refers to a polypeptide (or polynucleotide), that when expressed in a “Abph2 mutant line” that exhibits the “Abph2 mutant phenotype”, is capable of partially or fully rescuing the Abph2 mutant phenotype.

The terms “gene shuffling” and “directed evolution” are used interchangeably herein. The method of “gene shuffling” consists of iterations of DNA shuffling followed by appropriate screening and/or selection to generate variants of Abph2 nucleic acids or portions thereof having a modified biological activity (Castle et al., (2004) Science 304(5674): 1151-4; U.S. Pat. Nos. 5,811,238 and 6,395,547).

“TILLING” or “Targeting Induced Local Lesions IN Genomics” refers to a mutagenesis technology useful to generate and/or identify, and to eventually isolate mutagenised variants of a particular nucleic acid with modulated expression and/or activity (McCallum et al., (2000), Plant Physiology 123:439-442; McCallum et al., (2000) Nature Biotechnology 18:455-457; and, Colbert et al., (2001) Plant Physiology 126:480-484).

TILLING combines high density point mutations with rapid sensitive detection of the mutations. Typically, ethylmethanesulfonate (EMS) is used to mutagenize plant seed. EMS alkylates guanine, which typically leads to mispairing. For example, seeds are soaked in an about 10-20 mM solution of EMS for about 10 to 20 hours; the seeds are washed and then sown. The plants of this generation are known as M1. M1 plants are then self-fertilized. Mutations that are present in cells that form the reproductive tissues are inherited by the next generation (M2). Typically, M2 plants are screened for mutation in the desired gene and/or for specific phenotypes.

TILLING also allows selection of plants carrying mutant variants. These mutant variants may exhibit modified expression, either in strength or in location or in timing (if the mutations affect the promoter for example). These mutant variants may even exhibit lower ABPH2 activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H. Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (U.S. Pat. No. 8,071,840).

Other mutagenic methods can also be employed to introduce mutations in the Abph2 gene. Methods for introducing genetic mutations into plant genes and selecting plants with desired traits are well known. For instance, seeds or other plant material can be treated with a mutagenic chemical substance, according to standard techniques. Such chemical substances include, but are not limited to, the following: diethyl sulfate, ethylene imine, and N-nitroso-N-ethylurea. Alternatively, ionizing radiation from sources such as X-rays or gamma rays can be used.

Other detection methods for detecting mutations in the Abph2 gene can be employed, e.g., capillary electrophoresis (e.g., constant denaturant capillary electrophoresis and single-stranded conformational polymorphism). In another example, heteroduplexes can be detected by using mismatch repair enzymology (e.g., CELI endonuclease from celery). CELI recognizes a mismatch and cleaves exactly at the 3′ side of the mismatch. The precise base position of the mismatch can be determined by cutting with the mismatch repair enzyme followed by, e.g., denaturing gel electrophoresis. See, e.g., Oleykowski et al., (1998) “Mutation detection using a novel plant endonuclease” Nucleic Acid Res. 26:4597-4602; and, Colbert et al., (2001) “High-Throughput Screening for Induced Paint Mutations” Plant Physiology 126:480-484.

The plant containing the mutated Abph2 gene can be crossed with other plants to introduce the mutation into another plant. This can be done using standard breeding techniques.

Homologous recombination allows introduction in a genome of a selected nucleic acid at a defined selected position. Homologous recombination has been demonstrated in plants. See, e.g., Puchta et al. (1994), Experientia 50: 277-284; Swoboda et al. (1994), EMBO J. 13: 484-489; Offringa et al. (1993), Proc. Natl. Acad. Sci. USA 90: 7346-7350; Kempin et al. (1997) Nature 389:802-803; and, Terada et al., (2002) Nature Biotechnology, 20(10):1030-1034).

Methods for performing homologous recombination in plants have been described not only for model plants (Offringa et al. (1990) EMBO J. October; 9(10):3077-84) but also for crop plants, for example rice (Terada R, Urawa H, Inagaki Y, Tsugane K, Iida S. Nat Biotechnol. 2002; Iida and Terada: Curr Opin Biotechnol. 2004 April; 15(2):1328). The nucleic acid to be targeted (which may be ABPH2 nucleic acid or a variant thereof as hereinbefore defined) need not be targeted to the locus of ABPH2 gene respectively, but may be introduced in, for example, regions of high expression. The nucleic acid to be targeted may be weak Abph2 allele or a dominant negative allele used to replace the endogenous gene or may be introduced in addition to the endogenous gene.

Transposable elements can be categorized into two broad classes based on their mode of transposition. These are designated Class I and Class II; both have applications as mutagens and as delivery vectors. Class I transposable elements transpose by an RNA intermediate and use reverse transcriptases, i.e., they are retroelements. There are at least three types of Class I transposable elements, e.g., retrotransposons, retroposons, SINE-like elements. Retrotransposons typically contain LTRs, and genes encoding viral coat proteins (gag) and reverse transcriptase, RnaseH, integrase and polymerase (poi) genes. Numerous retrotransposons have been described in plant species. Such retrotransposons mobilize and translocate via a RNA intermediate in a reaction catalyzed by reverse transcriptase and RNase H encoded by the transposon. Examples fall into the TyI-copia and Ty3-gypsy groups as well as into the SINE-like and LINE-like classifications (Kumar and Bennetzen (1999) Annual Review of Genetics 33:479). In addition, DNA transposable elements such as Ac, TamI and En/Spm are also found in a wide variety of plant species, and can be utilized in the disclosure. Transposons (and IS elements) are common tools for introducing mutations in plant cells.

### EMBODIMENTS

In one embodiment, the Abph2 variant that can be used in the methods of the disclosure is one or more of the following ABPH2 nucleic acid variants: (i) a portion of a Abph2 nucleic acid sequence (SEQ ID NO: 2); (ii) a nucleic acid sequence capable of hybridizing with a Abph2 nucleic acid sequence (SEQ ID NO: 2); (iii) a splice variant of a Abph2 nucleic acid sequence (SEQ ID NO: 2); (iv) a naturally occurring allelic variant of a Abph2 nucleic acid sequence (SEQ ID NO: 2); (v) a Abph2 nucleic acid sequence obtained by gene shuffling; (vi) a Abph2 nucleic acid sequence obtained by site-directed mutagenesis; (vii) a Abph2 variant obtained and identified by the method of TILLING.

In one embodiment, the levels of endogenous Abph2 expression can be decreased in a plant cell by antisense constructs, sense constructs, RNA silencing constructs, RNA interference, and genomic disruptions. Examples of genomic disruption include, but are not limited to, disruptions induced by transposons, tilling, homologous recombination.

In one embodiment, a nucleic acid variant of Abph2 useful in the methods of the disclosure is a nucleic acid variant obtained by gene shuffling.

In one embodiment, a genetic modification may also be introduced in the locus of a maize Abph2 gene using the technique of TILLING (Targeted Induced Local Lesions In Genomes).

In one embodiment, site-directed mutagenesis may be used to generate variants of Abph2 nucleic acids. Several methods are available to achieve site-directed mutagenesis. In general, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom” meganucleases produced to modify plant genomes (see e.g., WO 2009/114321; Gao et al. (2010) Plant Journal 1:176-187). Another site-directed engineering is through the use of zinc finger domain recognition coupled with the restriction properties of restriction enzyme. See e.g., Urnov, et al., (2010) Nat Rev Genet. 11(9):636-46; Shukla, et al., (2009) Nature 459 (7245):437-41.

In one embodiment homologous recombination can also be used to inactivate, or reduce the expression of endogenous Abph2 gene in a plant.

Homologous recombination can be used to induce targeted gene modifications by specifically targeting the Abph2 gene in viva Mutations in selected portions of the Abph2 gene sequence (including 5′ upstream, 3′ downstream, and intragenic regions) such as those provided herein are made in vitro and introduced into the desired plant using standard techniques. Homologous recombination between the introduced mutated Abph2 gene and the target endogenous ABPH2 gene would lead to targeted replacement of the wild-type gene in transgenic plants, resulting in alteration of Abph2 expression or activity.

In one embodiment, catalytic RNA molecules or ribozymes can also be used to inhibit gene expression. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the constructs. A number of classes of ribozymes have been identified. For example, one class of ribozymes is derived from a number of small circular RNAs that are capable of self-cleavage and

replication in plants. The RNAs can replicate either alone (viroid RNAs) or with a helper virus (satellite RNAs). Examples of RNAs include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes has been described. See, e.g., Haseloff et al. (1988) Nature, 334:585-591.

In one embodiment, the Abph2 gene can also be activated by, e.g., transposon based gene activation.

In one embodiment, the inactivating step comprises producing one or more mutations in the Abph2 gene sequence, where the one or more mutations in the Abph2 gene sequence comprise one or more transposon insertions, thereby altering the Abph2 gene expression compared to a corresponding control plant. For example, the mutation may comprise a homozygous disruption in the Abph2 gene or the one or more mutations comprise a heterozygous disruption in the Abph2 gene or its regulatory element.

These mobile genetic elements are delivered to cells, e.g., through a sexual cross, transposition is selected for and the resulting insertion mutants are screened, e.g., for a phenotype of interest. Plants comprising disrupted Abph2 genes (i.e., modulated expression of Abph2 or its activity) can be crossed with a wt plant. Any of a number of standard breeding techniques can be used, depending upon the species to be crossed. The location of a TN (transposon) within a genome of an isolated or recombinant plant can be determined by known methods, e.g., sequencing of flanking regions as described herein. For example, a FOR reaction from the plant can be used to amplify the sequence, which can then be diagnostically sequenced to confirm its origin. Optionally, the insertion mutants are screened for a desired phenotype, such as the inhibition of expression or activity of Abph2 or alteration of an agronomic characteristic.

### EXAMPLES

The present disclosure is further illustrated in the following Examples, in which parts and percentages are by weight and degrees are Celsius, unless otherwise stated. It should be understood that these examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Furthermore, various modifications of the disclosure in addition to those shown and described herein will be apparent to those skilled in the art from the foregoing description. Such modifications are also intended to fall within the scope of the appended claims.

Sequence alignments and percent identity calculations were performed using the MEGALIGN® program of the LASERGENE® bioinformatics computing suite (DNASTAR® Inc., Madison, Wis.). Multiple alignment of the sequences was performed using the Clustal W method of alignment (Thompson, J. D., et al. (1994). Nucleic Acids Research, 22: 4673-80) with the default parameters (GAP PENALTY=10, GAP LENGTH PENALTY=0.2, DELAY DEVERGENT SEQS(%)=30%, DNA TRANSITION WEIGHT=0.5, PROTEIN WEIGHT MATRIX “Gonnet Series”).

Default parameters for pairwise alignments using the Clustal method were SLOW-ACCURATE, GAP PENALTY=10, GAP LENGTH=0.10, PROTEIN WEIGHT MATRIX “Gonnet 250”.

### Example 1

### Cloning of Maize Abph2 Gene

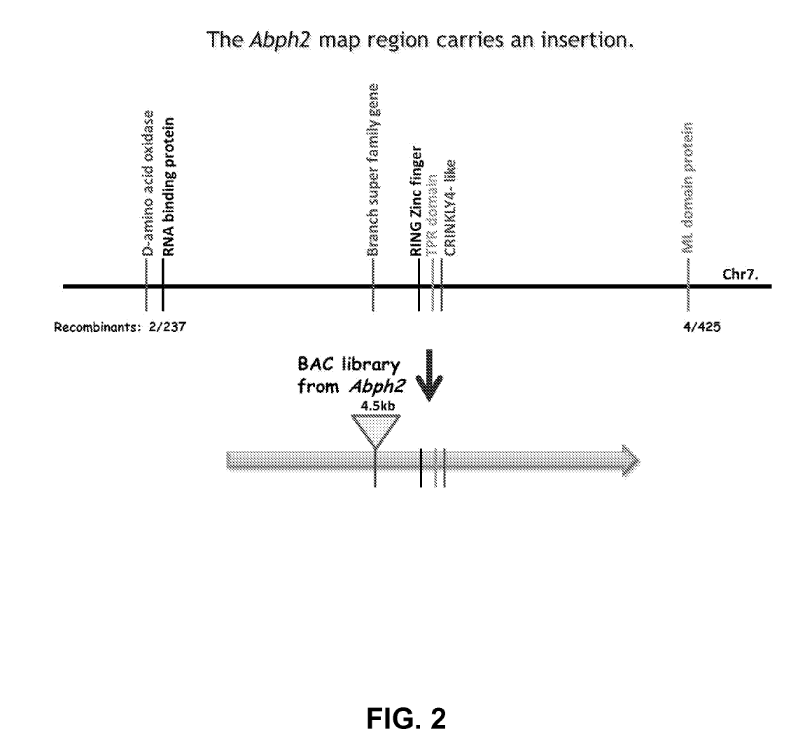

A map-based cloning approach was used to identify and isolate the Abph2 gene. Abphyl2 was initially mapped using a genome wide panel of SSR markers to the top of chromosome 7. Finer mapping using ˜50 individuals placed the mutation between markers mmc0171 and umc1577, and finer mapping using ˜1,000 individuals narrowed the region to between the predicted genes AC195322.2_FG002 on BAC b0226D18, and AC201967.3_FG002 on BAC b0316O18. (FIG. 2).

These genes are predicted to be on adjacent BAC clones, however the candidate genes in the region did not show any detectable changes in Abph2 mutants. Additional probes from these BACs were used to probe a BAC library that was made from the Abphyl2 mutant. A single BAC spanning the region was isolated and sequenced. When compared to the B73 reference genome, this BAC contained an insertion of ˜4.5 kb, which contained a single gene, a predicted glutaredoxin (GRX).

This GRX gene is present in the B73 reference genome but at a different location on Chr. 7, about 800 kbp away. Loss of function of that copy of the GRX gene leads to a male sterile phenotype (msca1, ms22, See e.g., U.S. Pat. No. 7,915,478).

### Example 2

### Expression Analysis of Abph2

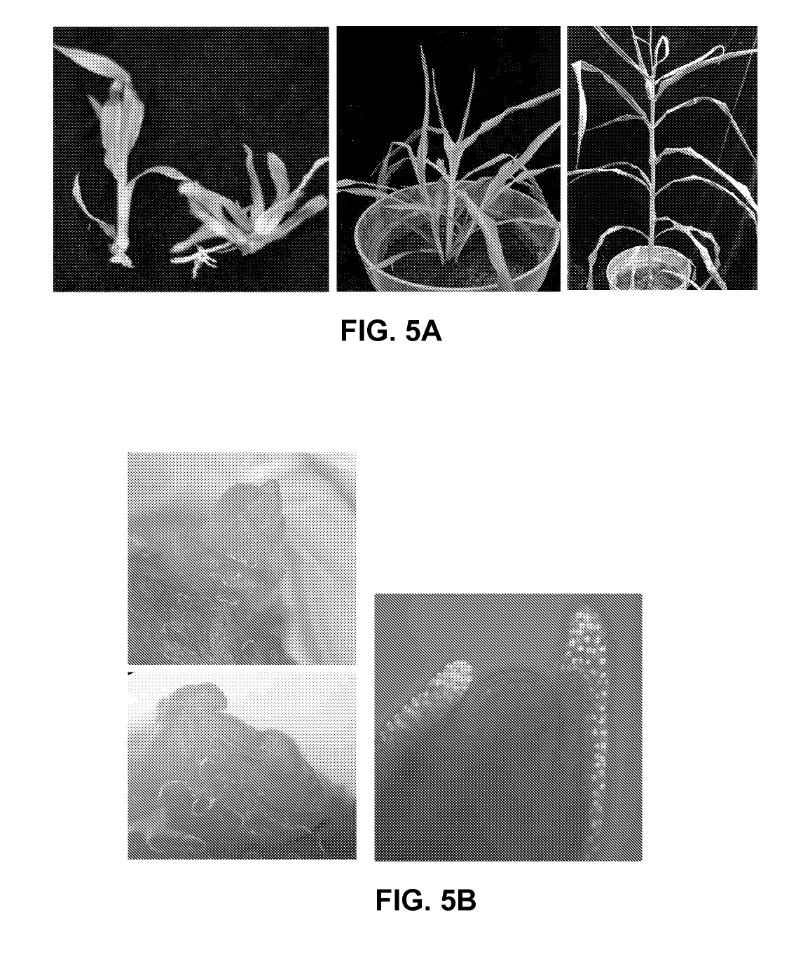

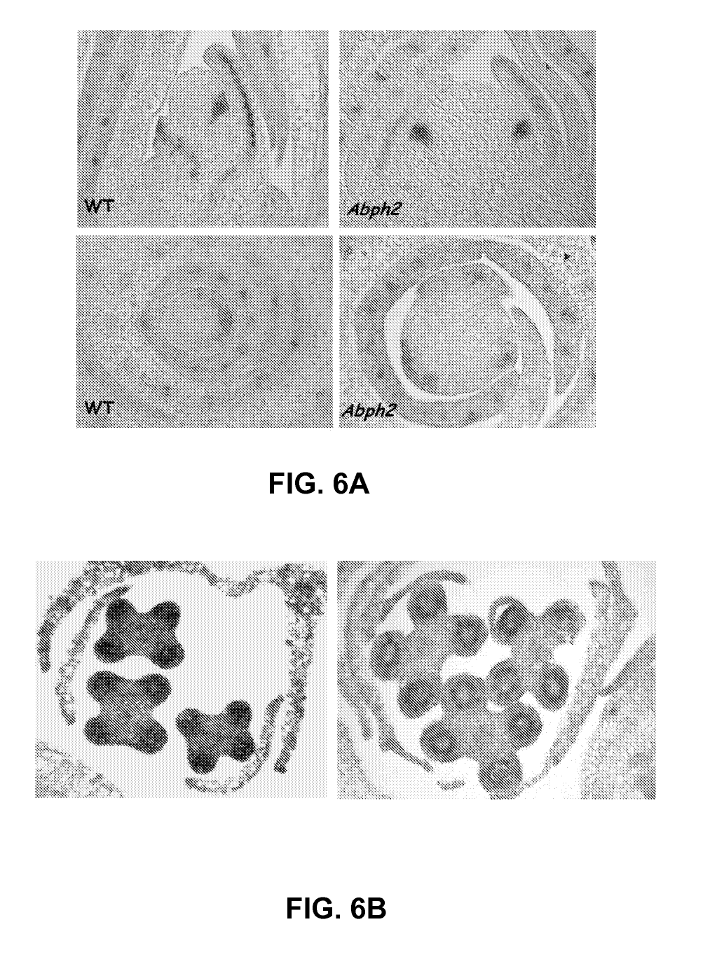

Abph2 is expressed in the shoot apical meristems in a localized pattern, in the domain of leaf initiation, and in leaf vascular tissues (FIGS. 5B and 6A). It is also expressed in developing anthers (FIG. 6B).

### Example 3

### Maize Mutant Abph2 Phenotype

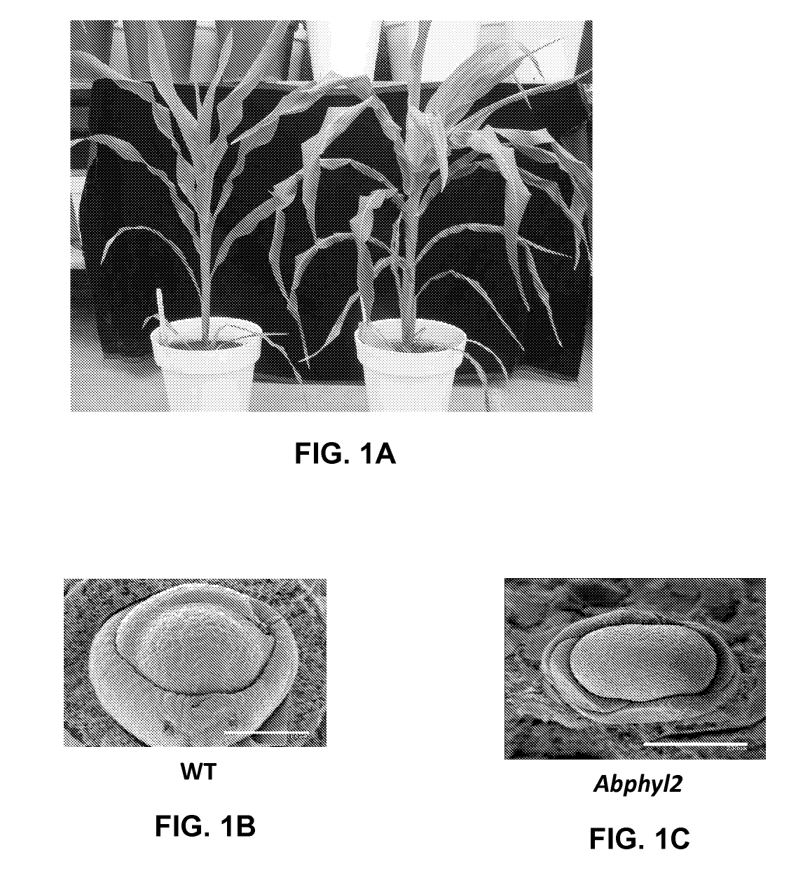

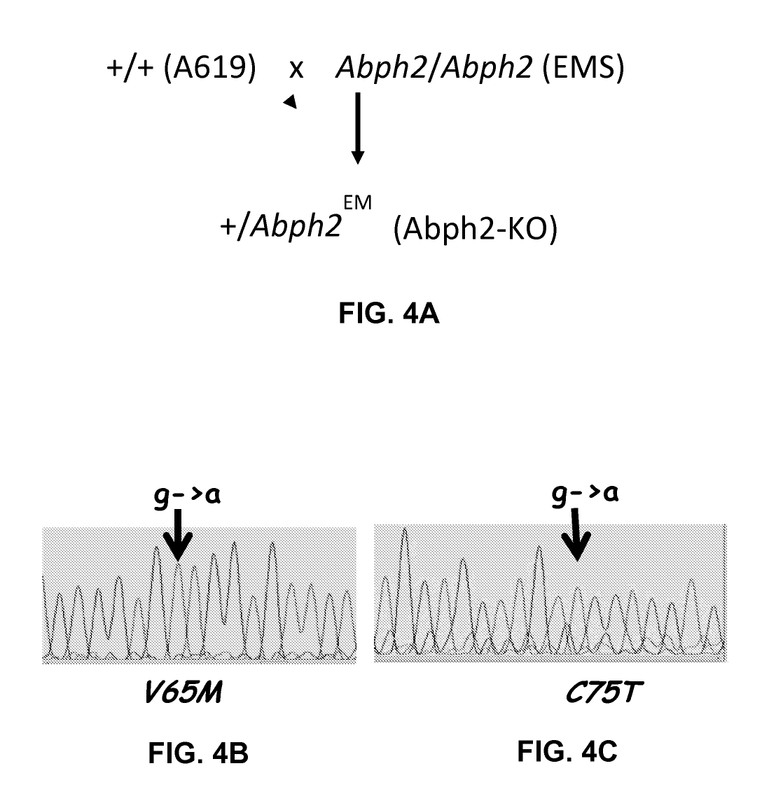

ABPHYL2 is a new dominant locus that controls the patterns of leaf initiation (“phyllotaxy”) in maize. In Abph2 mutants, leaves are made in opposite pairs, rather than one at a time as in wild type maize (FIGS. 1A and 5A). The mutants also produce two ears at a single node, instead of one, and therefore this phenotype may be used to increase yield in the field. Ahph2 was isolated by positional cloning and sequencing of a BAC from the mutant line, and the corresponding gene has been proven by sequencing of EMS induced genetic revertants (FIGS. 4A and 4B) and by maize transformation (FIG. 5A).

Ahph2 encodes a predicted glutaredoxin protein. Such proteins catalyze redox exchange reactions to form or break disulphide bonds in proteins. Based work in Arabidopsis, disclosed herein, it appears that Abph2 functions by catalyzing disulphide bonds between bZIP transcription factors.

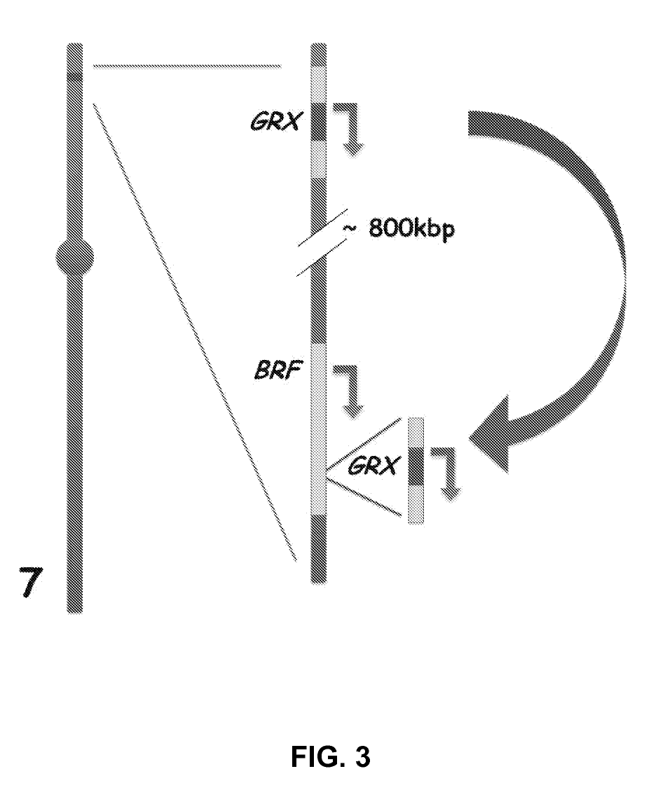

Abph2 is identical to the gene, msca1/ms22 gene disclosed previously (U.S. Pat. No. 7,915,478), where the homozygous recessive mutation caused male sterility. The Abph2 allele disclosed herein is dominant, and causes enlarged meristems and altered phyllotaxy. The disclosure provides a novel function for Abph2 gene in meristem development. The msca1/ms22 mutants do not have a meristem defect due to genetic redundancy. The Abph2 phenotype appears to have been caused by a translocation of the gene from its original location at the tip of chromosome 7 to a new location ˜800 kbp proximal (FIG. 3). This change may have introduced new regulatory elements to the endogenous gene, and therefore may cause a change in its expression during embryogenesis.

### Example 4

### Interaction with FEA4

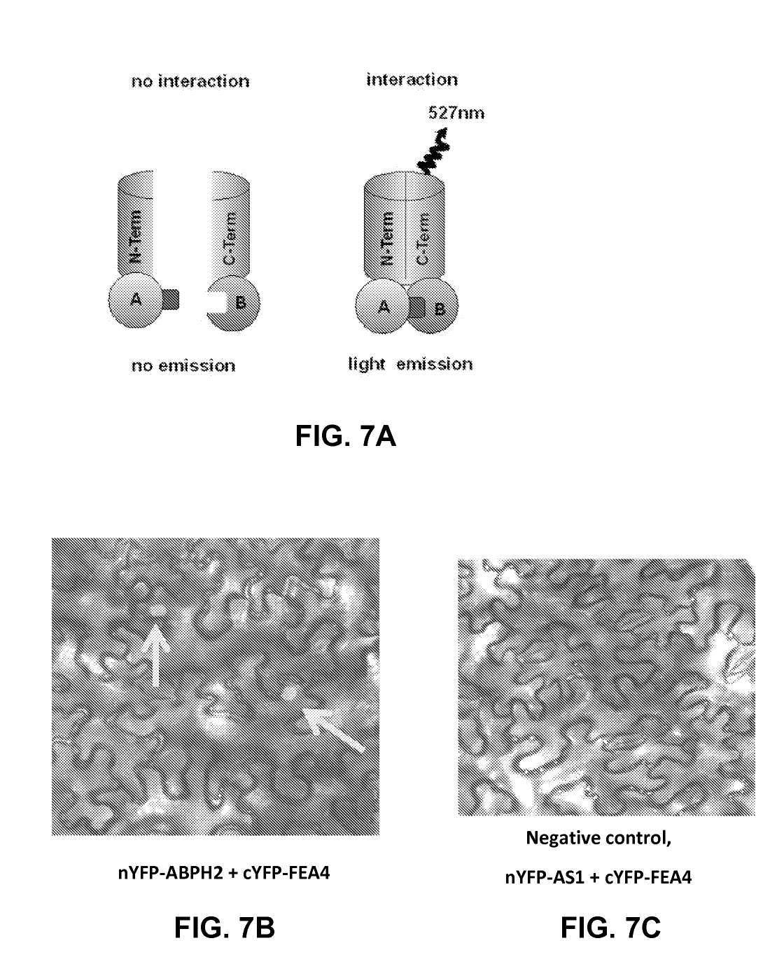

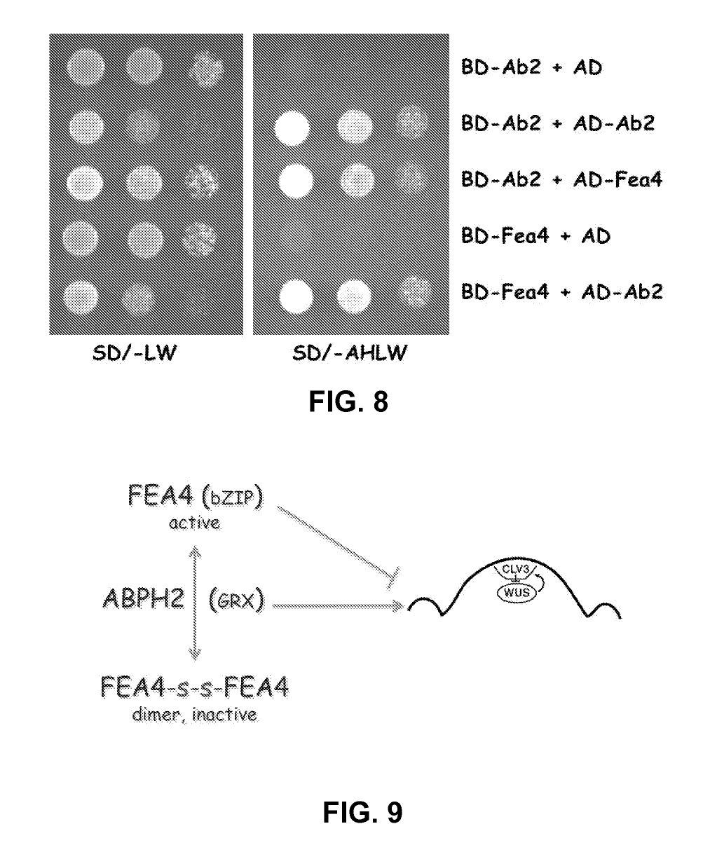

ABPH2 is shown to interact with a bZIP transcription factor FEA4 (U.S. Provisional Application No. 61/610,730, filed Mar. 14, 2012, titled “NUCLEOTIDE SEQUENCES ENCODING FASCIATED EAR4 (FEA4) AND METHODS OF USE THEREOF”. FIG. 7 shows that ABPH2 (GRX) and FEA4 (bZIP) interact in the nucleus. Interaction of FEA4 and ABPH2 by bimolecular fluorescence complementation is shown. FIG. 8 shows that ABPH2 (GRX) and FEA4 (bZIP interaction factor) interact via yeast 2 hybrid interaction. SD/−LW synthetic dropout media minus leucine and tryptophan. A meristem development model involving ABPH2 and FEA4 is shown in FIG. 9.

### Example 5

### Homologs of ABPH2

Sequences homologous to ABPH2 were identified using sequence comparison algorithms such as BLAST (Basic Local Alignment Search Tool; Altschul et al., J. Mol. Biol. 215:403-410 (1993); see also the explanation of the BLAST algorithm on the world wide web site for the National Center for Biotechnology Information at the National Library of Medicine of the National Institutes of Health). Public and proprietary databases were searched to obtain ABPH2 homolog sequences (SEQ ID NO: 6-31).

1. A method of producing a transgenic plant with modulated expression of Abph2, the method comprising:
a. introducing into a regenerable plant cell a recombinant construct comprising a polynucleotide operably linked to a promoter, wherein the expression of the polynucleotide sequence modulates Abph2 expression or activity;
b. regenerating a transgenic plant from the regenerable plant cell after step (a), wherein the transgenic plant comprises in its genome the recombinant DNA construct; and
c. selecting the transgenic plant of (b), wherein the transgenic plant comprises the recombinant construct and exhibits an alteration in the expression of Abph2, when compared to a control plant not comprising the recombinant DNA construct.

2. (canceled)

3. The method of claim 1, wherein the polynucleotide encodes an amino acid sequence selected from the group consisting of SEQ ID NOS: 1 and 6-31, a functional domain thereof, and a sequence that is at least 70% identical to SEQ ID NOS: 1 and 6-31.

4. (canceled)

5. (canceled)

6. A method of increasing an agronomic characteristic of a plant, the method comprising,
a. introducing into a regenerable plant cell a DNA construct comprising an isolated polynucleotide operably linked in sense orientation to a promoter functional in a plant, wherein the polynucleotide comprises:
i. the nucleotide sequence of SEQ ID NO: 2 or 5;
ii. a nucleotide sequence that encodes a polypeptide with the amino acid sequence of SEQ ID NO: 1 or a sequence that is at least 70% identical to SEQ ID NO: 1, based on the Clustal V method of alignment, when compared to SEQ ID 1;
iii. a nucleotide sequence that encodes the amino acid sequence selected from the group consisting of SEQ ID NOS: 1 and 6-31, a functional domain thereof, and a sequence that is at least 70% identical to SEQ ID NOS: 1 and 6-31; or
iv. a nucleotide sequence that can hybridize under stringent conditions with the nucleotide sequence of (i);
b. regenerating a transgenic plant cell after step (a), wherein the transgenic plant comprises in its genome the recombinant DNA construct; and
c. selecting a transgenic plant of (b), wherein the transgenic plant comprises the recombinant DNA construct and exhibits an alteration of at least one agronomic characteristic selected from the group consisting of: ear meristem size, kernel row number, leaf number, inflorescence number, branching within the inflorescence, flower number, fruit number, seed number, biomass and yield, when compared to a control plant not comprising the recombinant DNA construct.

7. A method of identifying an allele of Abph2, the method comprising the steps of:
a. performing a genetic screen on a population of mutant maize plants;
b. identifying one or more mutant maize plants that exhibit varying degrees of Abph2 phenotype;
c. identifying the Abph2 allele from the mutant maize plant with a varying Abph2 phenotype.

8. (canceled)

9. A plant in which expression of the endogenous Abph2 gene is altered relative to a control plant.

10. (canceled)

11. The plant of claim 9, wherein expression of the endogenous Abph2 gene is altered such that the Abph2 gene is altered in its expression during embryogenesis relative to a control plant.

12. (canceled)

13. (canceled)

14. A method of making the plant of claim 11, the method comprising the steps of
a. introducing a mutation into the endogenous Abph2 gene; and
b. detecting the mutation.

15. The method of claim 14, wherein using the steps (a) and (b) are done using Targeting Induced Local Lesions IN Genomics (TILLING) method and wherein the mutation is effective in altering the expression of the endogenous Abph2 gene or its activity.

16. The method of claim 14, wherein the mutation is a site-specific mutation.

17. A method of making the plant of claim 9, wherein the method comprises the steps of:
a. introducing an insertion into the endogenous Abph2 gene of a regenerable plant cell using a transposon;
b. regenerating a transgenic plant from the regenerable plant cell of step (a), wherein the transgenic plant comprises in its genome the transposon insertion;
c. selecting a transgenic plant from step (b) wherein the transgenic plant comprises in its genome the insertion of step (a) and exhibits an alteration in the expression of Abph2.

18. The method of claim 1, wherein said plant is selected from the group consisting of: Arabidopsis, tomato, maize, soybean, sunflower, sorghum, canola, wheat, alfalfa, cotton, rice, barley, millet, sugar cane and switchgrass.

19. A plant comprising in its genome a recombinant DNA construct comprising an isolated polynucleotide operably linked, in sense orientation to a promoter functional in a plant, wherein the polynucleotide comprises a nucleotide sequence that encodes the amino acid sequence selected from the group consisting of SEQ ID NOS: 1 and 6-31, a functional domain thereof, and a sequence that is at least 70% identical to SEQ ID NOS: 1 and 6-31, wherein the plant exhibits an alteration in at least one agronomic characteristic selected from the group consisting of: enlarged ear meristem, kernel row number, seed number, biomass and yield, when compared to a control plant not comprising the recombinant DNA construct.

20. The plant of claim 19, wherein said plant is selected from the group consisting of: Arabidopsis, tomato, maize, soybean, sunflower, sorghum, canola, wheat, alfalfa, cotton, rice, barley, millet, sugar cane and switchgrass.

21. (canceled)

22. (canceled)

23. (canceled)

24. A plant comprising in its genome a recombinant DNA construct comprising a Abph2 polynucleotide operably linked to a second polynucleotide, wherein the second polynucleotide is a shoot apical meristem-preferred promoter and wherein the heterologous polynucleotide is expressed in the plant.

25. The plant of claim 24, wherein said plant is selected from the group consisting of: Arabidopsis, tomato, maize, soybean, sunflower, sorghum, canola, wheat, alfalfa, cotton, rice, barley, millet, sugar cane and switchgrass.

26. (canceled)

27. (canceled)

28. (canceled)

29. (canceled)

30. (canceled)

31. An isolated polynucleotide encoding an amino acid sequence selected from the group consisting of SEQ ID NOS: 6-31, a functional domain thereof, and a sequence that is at least 70% identical to SEQ ID NOS: 6-31.

32. The polynucleotide of claim 31 is recombinant.

33. The polynucleotide of claim 31 is expressed in a heterologous host.

34. A method of making the plant of claim 24, the method comprising:
a. transforming a regenerable plant cell with a recombinant DNA construct comprising a Abph2 polynucleotide operably linked to a second polynucleotide, wherein the second polynucleotide is a shoot apical meristem-preferred promoter
b. regenerating a transgenic plant from the regenerable plant cell after step (a), wherein the transgenic plant comprises in its genome the recombinant DNA construct; and
c. selecting a transgenic plant of (b), wherein the transgenic plant comprises the recombinant DNA construct and further wherein the heterologous polynucleotide is expressed in the transgenic plant.

35. The method of claim 34, wherein said plant is selected from the group consisting of: Arabidopsis, tomato, maize, soybean, sunflower, sorghum, canola, wheat, alfalfa, cotton, rice, barley, millet, sugar cane and switchgrass.


SEQUENCE LISTING (31 sequences)

SEQ ID NO: 1 - protein, 159 aa, Zea mays
MLRMEVQQQQQESGVSGGVVADAAAASGADAAPTTTTMVAAAPHSASALAVYERVARMAGGNAVVVFSASGCCMCHVVKRLLLGLGVGPTVYELDQMAAGGGGGREIQAALAQLLPPGQPPLPVVFVGGRLLGGVEKVMACHINGTLVPLLKQAGALWL

SEQ ID NO: 2 - DNA, 486 bp, Zea mays
acgaagatgctgcggatggaggtgcagcagcagcagcaggagtcgggagtgagcggcggcgtggtggcggacgcggcggcggcatccggggcggatgccgcgccgacgacgacgacgatggtggccgcggcgccgcactcggcgtcggcgctggcggtgtacgagcgggtggcgcgcatggcgggcgggaacgcggtggtggtgttcagcgccagcggctgctgcatgtgccacgtcgtcaagcgcctgctgctgggcctcggcgtcggccccaccgtgtacgagctcgaccagatggccgccggcggcggcgggggcagggagatccaggcggcgctggcgcagctgctgccgccgggccagccgcccctgcccgtcgtcttcgtgggcggacgcctcctcggcggcgtcgagaaggtcatggcgtgccacatcaacggcaccctcgtcccgctcctcaagcaggccggcgcgctctggctctga

SEQ ID NO: 3 - DNA, 480 bp, Zea mays
  misc_feature (193)..(193): G193A change results in V65M mutation
atgctgcggatggaggtgcagcagcagcagcaggagtcgggagtgagcggcggcgtggtggcggacgcggcggcggcatccggggcggatgccgcgccgacgacgacgacgatggtggccgcggcgccgcactcggcgtcggcgctggcggtgtacgagcgggtggcgcgcatggcgggcgggaacgcggtgatggtgttcagcgccagcggctgctgcatgtgccacgtcgtcaagcgcctgctgctgggcctcggcgtcggccccaccgtgtacgagctcgaccagatggccgccggcggcggcgggggcagggagatccaggcggcgctggcgcagctgctgccgccgggccagccgcccctgcccgtcgtcttcgtgggcggacgcctcctcggcggcgtcgagaaggtcatggcgtgccacatcaacggcaccctcgtcccgctcctcaagcaggccggcgcgctctggctctga

SEQ ID NO: 4 - DNA, 480 bp, Zea mays
  misc_feature (224)..(224): G224A change results in C75T substitution
atgctgcggatggaggtgcagcagcagcagcaggagtcgggagtgagcggcggcgtggtggcggacgcggcggcggcatccggggcggatgccgcgccgacgacgacgacgatggtggccgcggcgccgcactcggcgtcggcgctggcggtgtacgagcgggtggcgcgcatggcgggcgggaacgcggtggtggtgttcagcgccagcggctgctgcatgtaccacgtcgtcaagcgcctgctgctgggcctcggcgtcggccccaccgtgtacgagctcgaccagatggccgccggcggcggcgggggcagggagatccaggcggcgctggcgcagctgctgccgccgggccagccgcccctgcccgtcgtcttcgtgggcggacgcctcctcggcggcgtcgagaaggtcatggcgtgccacatcaacggcaccctcgtcccgctcctcaagcaggccggcgcgctctggctctga

SEQ ID NO: 5 - DNA, 4518 bp, Zea mays
  misc_feature (1)..(4518): This region is an insertion in the maize genome
agtaataaatcctagtgttgtacagcataggtagccgccactcctgctctgtgggtttcgtgtctcgatgtttttgtactttgcagtgtactacgtacatactaagatgtgcttccaacttccatccagttaaaagttgagcagtttgtggtctctcctgcgtttaccagatgtgctgggcactggacgcgcgtcggtcgtcggcacacaccaacaaaaaaacactgaagcacatctgcatgtatttttgcgcgcaaagatacgctcgtggcaacagagagaggtgcacagagatactaccaggagcctcgatatgtacaccgaagcacatctgcatgtaagcaggagcctggatatgtattttttgcgcaggtggcacgcatgcagctgtgctgtgctgtgctgttcgctgtgcacggctagcaggagcccacacgtgtgcggcatgcacgccgccgccgagcgatcggatccgagggcagaaagcactgacagcacagcgcaggccaggaggggtctgcctctgtctgatcgacgatcctccggccgctctcgctgcgcgtgcggctgaatgccactgccatgccgctgcaggcagcagctgccgccgtgcgtggtcctggctcgctacactgtctcgagtctcgacaaatgaaaacaccagttgaccatctcctctctctctaggctctaggctctagctcgcatgcgtctcttctgtgtattgtaacgctgttcgatgtggcctggtactaagctccgtagtccgtacagctatcagaggtctttcacatgatcacgtcgtcggctccgatccatcacatccacagcacaagcaccatgtggcgacaccgtggctcacgggcttcaaacccaactttaggatatcaaatctttgttgcattgcattgcacgccgctgctaaccatgtatactcactacctgactaccagagcgagaatgaaaaggggcccagcccatatgaatgataatgaacccaaaacgtcacgtcggcccatccgtccagcttgcatcgagtcggcctcgaacgaccgagggaggggaagaagtcgccgtcgcccgcatcgcagagcgagcaaatccaatgggctatggggatacgggcctacggcgaagtgctgcacaccacgcgggctgctggaaatcttgcgactctacttcctctttgtcactgtatctgtcgctccagcgtgagcctggcctgacgcatttacaccgtagtacatgaccgtgcttaattactgtcgctctcctccggcagcaatagccacatcgacaaaatgaccctaatggaccttaaagtactttaaaggactcattaaactttatcaaagtcatacaacaaaatccgcaagacaaacaaacaaaaaaacaaacttacaaaaccacaccgtatttatctggatggtgggcgagtctttttaatcaccatggcagacttactcttgttaatgcagtcttggacagcttgcctgtttatgcaataggggctctcgccctccctccaggcgtgattgaggccatcgactctagacgtcgggctttcctatgggtcggggaagaaacagtctctcgtgcgaagtgtcttgtgaattgggaacggacatgtctcccgaaaaaagatggcggactgggtgttcgtgatctccgcttgcagaatacctgtctactactgaagctcctccatcgtgcacacaactcacgtgactcagcttgggcccgctggttagaagtggaatttggaggccccatgtctgctccagacaacacggccaacgaagtgtatgtacatatatacccatggtcatatggcaacaaacgccaacgccagcagagcactgcccggcggcctttttcccatctctctctctctctctgatggggtgtgcatgcctgactgactgatagatagatagatggtcaggtccgtctgatcctcatcggcctagctcaccccacgcgaaaaaagccactgctggctggcgcccagttgcgcttgcaacagtcactttaacgagctccgtccttgcgtttgccctcctcgctctgccccctggctccccgccgctgcgtggtggtgctggtgcatgaggcaggcgtactagtgcatgcaatgcaaccgtaggagtgcgttgcgtaccctggtctgtccctgcggcctggcctgccgcccttgttcgttgcggatgcggggggtgccgggtgggtactgtactgtactactgggtagagagaagtagatatagatagatagagagagagagaggtcggtcaccccgggcgcgggacacagcctctgcgaaaaagcgatccatgtcgcgcctagctttgacccggaacggatcccccaagtccccaaccaggaaccagcagagcaggagggccaggccaggccaccacctctctcgccattccattcccggtcctagctagtcctgttctgttcctgtagcagcagtagcagtagctacggtactacgagtcctcctcgacgtcccaggcactactccacgcagcagcaggcagcggcgagcatctctcgaccagatgcatacaagctacaccctcctcggctccgatcctacccatgccggcccaggcgtcctataaaagcgcacccccggcccgtcttcctcccactgcaatactgcatgcccatcacccccttcgccgtgccaacgacacacctcatcaccggccggaacattccacgaccgaagaaaccagtccctagctagtccacgcacgaccaacaaggcaggcgagcgacgacagtccaaagcctccaagaagaagaagaacgaagatgctgcggatggaggtgcagcagcagcagcaggagtcgggagtgagcggcggcgtggtggcggacgcggcggcggcatccggggcggatgccgcgccgacgacgacgacgatggtggccgcggcgccgcactcggcgtcggcgctggcggtgtacgagcgggtggcgcgcatggcgggcgggaacgcggtggtggtgttcagcgccagcggctgctgcatgtgccacgtcgtcaagcgcctgctgctgggcctcggcgtcggccccaccgtgtacgagctcgaccagatggccgccggcggcggcgggggcagggagatccaggcggcgctggcgcagctgctgccgccgggccagccgcccctgcccgtcgtcttcgtgggcggacgcctcctcggcggcgtcgagaaggtcatggcgtgccacatcaacggcaccctcgtcccgctcctcaagcaggccggcgcgctctggctctgatcgcgccgccgccgtcgtcgtcgtcgatcggccactgcaacagtgtgtgtgtgtgggtgtccatctccgtgcatgcgatcgatcgctgccccttagaccttagttagttactcacttactaccttagcttgcgttttaatgtaacctctactaagctagctagctcttgttctgttccgtgcccatgcatgagagagatcgagtaatgctgcaatcgcctgctgcaattaatgcagcagcgcacgacgtcgccgatatatgatggtgcatcgattattgcactccgatccatggatatcatcgatcttaaccggacgtggacgtacggtgccccggccggtgcaggcagatcgaggagggcggcggccggcgatgcccccggctgaggtcacagagcagcagcagggggccagtcagcagccttgtaaaagcgtacgtacgtacgtacgtcgtcgagacatcaacgacgtacggggacgcaacgcaaccagccaaaacgggatcgttcgaactagagcaagacgtacggcttttgatgagcttgcggtgttagactgttagacataaaaaaatacataatataataaacatacaagctatccatggtttctagctttatgcatgttggggctgcactactgaccatagtaattcgctcatcaagtgttgtgaggtaccatggaaacttcaaactacgtacagttagcaaatatggaccaaacacaatgcacaaaattttacgtgaaaagactttataggaagaaaaaacacgagcgtcaatcgtcaatcttcattatacgatgagagcttatagaacatagaaaataccttctcgtgcgacttataagttgtatatatatacgacggacaaactatagtctaatatgaaaataaatccactaattgatataacccgacctgcacatgtgcctccagacgtcctcgcaagactcgcgctaaaattcacacgcaagactcgcgctagaattcacattgtatttcaataaactccacctcatggagacggcggcggtgcatcagcaggcagaggagggcgcgacggcgacgctcccccgggcgggccccaagctgatgtcagggagcaggagccagagcagccttgagctgggcctgggcagctggcgatcgagcgtccctttgtaaaaaaaaataaataaaaaagtggcttttgacgggccagctgatggctt

SEQ ID NO: 6 - protein, 192 aa, Oryza sativa
MSERVFAELATIHYQKSLPCRHSFDPPRTTPILHLYIIHLLLPPLIAIVCLCYIAIVPFEEEEERMRMQVVETAAVEEEEAAAAMMSVYERVARMASGNAVVVFSASGCCMCHVVKRLLLGLGVGPAVYELDQLAAAADIQAALSQLLPPGQPPVPVVFVGGRLLGGVEKVMACHINGTLVPLLKQAGALWL

SEQ ID NO: 7 - protein, 166 aa, Sorghum bicolor
MLRMELQQAESGVSAGAGGVADAVADADAMMMVVSAAPPHSHHAAHPPPSAPLAVYERVARMASANAVVVFSASGCCMCHVVKRLLLGLGVGPTVYELDQMMAAAGPGGGGREIQAALAQLLPPGQPPVPVVFVGGRLLGGVEKVMACHINGTLVPLLKQAGALWL

SEQ ID NO: 8 - protein, 140 aa, Arabidopsis thaliana
MQYKTETRGSLSYNNNSKVMNNMNVFPSETLAKIESMAAENAVVIFSVSTCCMCHAIKRLFRGMGVSPAVHELDLLPYGVEIHRALLRLLGCSSGGATSPGALPVVFIGGKMVGAMERVMASHINGSLVPLLKDAGALWL

SEQ ID NO: 9 - protein, 136 aa, Arabidopsis thaliana
MQYQTESWGSYKMSSLGFGGLGMVADTGLLRIESLASESAVVIFSVSTCCMCHAVKGLFRGMGVSPAVHELDLHPYGGDIQRALIRLLGCSGSSSPGSLPVVFIGGKLVGAMDRVMASHINGSLVPLLKDAGALWL

SEQ ID NO: 10 - protein, 102 aa, Arabidopsis thaliana
MENLQKMISEKSVVIFSKNSCCMSHTIKTLFLDLGVNPTIYELDEISRGKEIEHALAQLGCSPTVPVVFIGGQLVGGANQVMSLHLNRSLVPMLKRAGALWL

SEQ ID NO: 11 - protein, 102 aa, Arabidopsis thaliana
MENLQKMISEKSVVIFSKNSCCMSHTIKTLFLDFGVNPTIYELDEINIGREIEQALAQLGCSPTVPVVFIGGQLVGGANQVMSLHLNRSLVPMLKRAGALWL

SEQ ID NO: 12 - protein, 134 aa, Glycine max
MHYQAAAASWGSYVAGAPRNSAAAAVVVGDPLERIERLASESAVVIFSVSTCCMCHAIKRLFCGMGVNPTVHELDEDPRGKDLERALMRLLGTPSVVPVVFIGGKLVGTMDRVMACHINGTLVPLLKEAGALWL

SEQ ID NO: 13 - protein, 132 aa, Glycine max
MHYQAAAAAWWGSYVAAPRNAAAAVVGDPLERIERLASESAVVIFSVSTCCMCHAIKRLFCGMGVNPTVHELDEDPRGKDLERALMRLLGTPSVVPVVFIGGKLVGTMDRVMACHINGTLVPLLKEAGALWL

SEQ ID NO: 14 - protein, 129 aa, Sorghum bicolor
MQYAAAEQAWYMPAATTMAESAVARVERLASESAVVVFSVSSCCMCHAVKRLFCGMGVHPTVHELDLDPRGRELERALARLLGYGPAGAPVVPVVFIGGKLVGAMDRVMAAHINGSLVPLLKEAGALWL

SEQ ID NO: 15 - protein, 128 aa, Osmunda cinnamomea
MQCYVSDMSGRRTSLNLSDDSDSSPMETIERLAGENAVVVFSLSSCCMCHVVKRLFCSLGVNPTVYELDEEMGGQELKDALVRLVGDGQPVPAVFVGGKLVGGLDRVMAAHISGALVPLLKEAGALWL

SEQ ID NO: 16 - protein, 128 aa, Zea mays; identical to SEQ ID NO: 15

SEQ ID NO: 17 - protein, 140 aa, Momordica charantia
MQGLRRCSAVDVVHLDLSPPPTAASAASLSIDVAESAETRIRRLISEHPVIIFSRNSCCMCHVMKKLLATIGVHPTVIELDDHEIDALASCSSSSAPPSNNSAPAVFIGGALVGGLESLVALHLSGHLVPKLVEVGALWV

SEQ ID NO: 18 - protein, 105 aa, Viola soraria
MDRVTKLASQKAVVIFSKSSCCMSHAIKRLFYEQGVSPSIIELDQVESRSGKEMELALMRHFGCNPSVPAVFVGGKFVGSENTVMTLHLNGSLKELLKEAGALWL

SEQ ID NO: 19 - protein, 129 aa, Nepeta racemosa
MRQRSYHSFQYSDNFSAKGGAAPVDPAERIMRLASANAVVIFSRRSCFLSHAVKQLFCGMGVNAMVYELDERPRGRELERALMRLLGGGAAVPVVFVGGKLVGGIERVIASHINGTLVPLLKEAGALWL

SEQ ID NO: 20 - protein, 132 aa, Lamium amplexicaule
MQKALQYRDWLSSPAAAPPGDHDSGGGDAVNVRKLVAENAVVVFAREGCCMCHVIKLLLNGHGVNPVILDVDDHNERDVTTELIGIIGSGEALQFPAVFVGGELFGGLEQVMGARISGELVPRLREARALWL

SEQ ID NO: 21 - protein, 100 aa, Lamium amplexicaule
MEKVQKLASENGILIFSKSTCCLCYAVQILFKELRVNPRIFEIDQNPEGKEIEKALTRMGCGGPLPAVFVGGKLVGSTNEIMSLHLSGSLTPLLRQYQPN

SEQ ID NO: 22 - protein, 124 aa, Lamium amplexicaule
MGSLFSSTIKREDIDMALAKAKQIVSSNPVVVFSKTYCGYCSRVKQLLSQLEATHKVIELDEESDGDEIQAALHEWTGQRTVPNVFINGKHIGGSDVVISKHQQGKLVPLLAEAGAIAKLTAQT

SEQ ID NO: 23 - protein, 137 aa, Artemisia tridentate
MQQAIPYKAWTLPLDHSHVGNNNPTISAKGAVREAVSDNAVIVLARKGCCMSHVVKRLLISHGVNPSVFEFEEAEEKDVVKELEIITAEKGALVKPRVQFPAVFIGGKMYGGLDQVMATHITGELIPVLKQAGALWL

SEQ ID NO: 24 - protein, 99 aa, Artemisia tridentate
MDRVKSLASKSAAVIFTKSTCCMSHSIKTLFYELGASPAIHEVDHDADMESALRRLGCNPAIPAVFIGGKYIDSAKDVISLHVDGSLKQKLVEAKAIWF

SEQ ID NO: 25 - protein, 102 aa, Amaranthus hypochondriacus
MERVNELAKSKAAVIFAKSSDCMCHSIKTLFYDLGASPTVYEIDYDPKGREMEIALQRLGCMPSVPAIFIGGNFVGSAKDVLAAHLSGSLKNMLIAAKAIWL

SEQ ID NO: 26 - protein, 105 aa, Amaranthus hypochondriacus
MDKVVDKVTRIASERGVVIFAKSSCCLCYAVNILFQELGATPFIYEVDKDPEGKEIEKVLMKLGSQSALPVVFIGGKLMGSTNEIMSLHLAGQLVPLLRPHGVCN

SEQ ID NO: 27 - protein, 102 aa, Amaranthus hypochondriacus
MERVNELAKSKAAVIFAKSSDCMCHSIKTLFYDLGASPAVYEIDRDPRGREIETALQRLGCKPTVPAIFIGGKFVGSAKDVLAAHLTGSLKDMLIAAKAIWF

SEQ ID NO: 28 - protein, 162 aa, Sesbania bispinosa
MHQAIPYRSWRPLHNPTTHFTPQPLITLSHITDDDNNNNKRKRLSFLSSPKSLATTKKGMVLNMVSENAIIVFGRRGCCMSHVVKRLLLGLGVNPAVYEVEEKDEVDVTRELETIIGEKQGKVQFPTVFIGGKLFGGLDRIMATHISGELVPILKEAGALWL

SEQ ID NO: 29 - protein, 152 aa, Dennstaedtia punctilobula
MQCSCDSHITSNQACTSRRSSSIRNSKGEKLERLAGENAVVVFTLSSCCMCDVVCRLLCSLGVNPAIHQVDDDQELQRSLLSLPLCNNMQSELHSCPSSPSSSSSSPSPSSSLIVPAVFVGGKLLGGLDKVMAAHINGSLVPLLKDAGALWL

SEQ ID NO: 30 - protein, 149 aa, Dennstaedtia punctilobula
MQAQVQQPMAASSSAIPYTDLPRRREGAAGNRRSYTSNSSSPSTSCKSEVEGSSTQEWGMKSIASMENAVVVVSTKGCCMTHVVTRLLCSLGVNPCVVELEDEVLQAQQSNIPAVFIGGRLLGGVDTLLAAHITGSLVPQLKEAGALWL

SEQ ID NO: 31 - protein, 103 aa, Paspalum notatum
MDRVTKLASQRAVVIFSTSSCCMSHTVTRLLRELGVNPTVVELDEDPRGKEMEKALARLLGRSPAVPAVFIGGRLVGSTDKVMSLHLSGSLVQLLRNAGALWV